(19) United States
(12) Patent Application Publication
(10) Pub. No.: US 2016/0039742 A1
(43) Pub. Date: Feb. 11, 2016

(54) EXTRACTION OF TRAMADOL FROM NAUCLEA LATIFOLIA SMITH

(71) Applicant: UNIVERSITE JOSEPH FOURIER - GRENOBLE 1, St Martin D'heres (FR)

(72) Inventors: Michel DE WAARD, Grenoble (FR); Ahcène BOUMENDJEL, Grenoble (FR); Germain TAIWE SOTOING, Buea (CM)

(21) Appl. No.: 14/780,429

(22) PCT Filed: Mar. 26, 2014
(86) PCT No.: PCT/EP2014/056058

§ 371 Date: Sep. 25, 2015

(30) Foreign Application Priority Data
Mar. 26, 2013 (EP) 13305374.4

(51) Int. Cl. C07C 213/10

(52) U.S. Cl. CPC C07C 213/10; C07C 2101/14

(57) ABSTRACT

A process for obtaining (±)-cis-2-dimethylaminomethyl-1-(3-methoxyphenyl)-cyclohexanol includes the following steps: (a) extracting from roots of Nauclea Latifolia with an appropriate solvent, leading to a crude residue, and (b) purifying the crude residue to obtain a purified residue containing (±)-cis-2-dimethylaminomethyl-1-(3-methoxyphenyl)cyclohexanol.

The present invention concerns a process for extracting (±)-cis-2-dimethylaminomethyl-1-(3-methoxyphenyl)cyclohexanol, commercialized under the trade name tramadol, from a natural source, Nauclea Latifolia Smith (Rubiaceae).

The nomenclature used in the present application follows the rules of IUPAC nomenclature.

Compound 2-dimethylaminomethyl-1-(3-methoxyphenyl)cyclohexanol can be in the form of two isomers, the cis-isomer and the trans-isomer. Each isomer can be in the form of two enantiomers since each isomer contains two asymmetric carbon atoms. The cis-isomer (according to IUPAC nomenclature) can be in the form of (+)-cis-2-dimethylaminomethyl-1-(3-methoxyphenyl)cyclohexanol or (−)-cis-2-dimethylamino-methyl-1-(3-methoxyphenyl)-cyclohexanol. The racemic mixture of these two enantiomers is commercialized under the name tramadol.

Tramadol is a well-known commercial analgesic drug, which was manufactured for the first time by Grunenthal GmbH (Germany) and used for the treatment of moderate to severe pain.

Up to now, tramadol was usually synthesized by a Grignard reaction between 2-dimethylaminomethylcyclohexanone and 3-methoxyphenyl magnesium halide, as described in U.S. Pat. No. 6,652,589, which produces a mixture of cis- and trans-isomers of 2-dimethylaminomethyl-1-(3-methoxyphenyl)cyclohexanol and side products.

The isomer mixture may be chemically treated to separate the two isomers. The traditionally used method disclosed in the patents of Grünenthal GmbH, such as U.S. Pat. No. 3,652,589, involves steps, reflux of the hydrochloride with dioxane and filtration to obtain a solid cake essentially comprising cis-isomer.

EP 0 831 082 discloses a method for isolating tramadol, which involves a reaction of the isomer mixture with an electrophilic reagent, such as acetic anhydride or thionyl chloride, under such conditions that only the trans-isomer reacts and the cis-isomer remains intact. The desired cis-isomer is then easily separated by recrystallization from an appropriate solvent.

WO 99/36390 discloses another method of separation comprising contacting the isomer mixture with hydrobromic or hydriodic acid to form a salt thereof, and subjecting the salt to a re-crystallization step to obtain the isomerically pure tramadol hydrobromide or hydriodide, from which a preferred pharmaceutically acceptable form of tramadol, such as tramadol hydrochloride, is obtainable.

Nevertheless, these methods of purification may be costly and do not necessarily provide tramadol with very high purity.

Surprisingly, it has been found that (±)-cis-2-dimethylaminomethyl-1-(3-methoxyphenyl)cyclohexanol is present in Nauclea Latifolia Smith (Rubiaceae), a shrub commonly occurring in tropical Africa, widely used in traditional medicine to treat various diseases and affections, such as malaria, fever and pain.

Disposing of a natural source of tramadol represents a great advantage since it allows the preparation of tramadol by carrying out an extraction. This process thus considerably facilitates the medical access to this widely prescribed pain-killer drug.

The aim of the present invention is thus to provide with a simple and cost-effective process for extracting tramadol with a satisfactory purity from Nauclea Latifolia.

The present invention thus relates to a process for obtaining (±)-cis-2-dimethylaminomethyl-1-(3-methoxyphenyl)cyclohexanol comprising the following steps:
a) extraction from roots of Nauclea Latifolia with an appropriate solvent, leading to a crude residue, and
b) purification of said crude residue to obtain a purified residue containing (±)-cis-2-dimethylaminomethyl-1-(3-methoxyphenyl)cyclohexanol.

The term “tramadol” designates within the present description (±)-cis-2-dimethylaminomethyl-1-(3-methoxyphenyl)cyclohexanol.

As mentioned above, tramadol exists in the form of two enantiomers, which can be designated by (R,R)-tramadol and (S,S)-tramadol, respectively of following formulae:

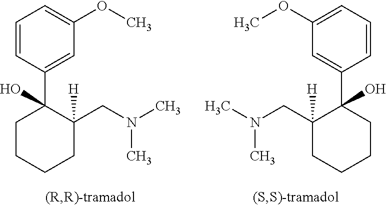 [SMILES: COc1cccc([C@@]2(O)CCCC[C@@H]2CN(C)C)c1 ; COc1cccc([C@]2(O)CCCC[C@H]2CN(C)C)c1]

In the present application, the name “Nauclea Latifolia” refers to the sub-Saharan Nauclea Latifolia (Rubiaceae) plant, commonly known as African peach or pin cushion tree. This plant is traditionally used by local populations for the treatment of several diseases. Indications include the treatment of severe digestive affections, neurological disorders, and infectious diseases. In Cameroon, the plant is used to treat pain, malaria, fever and infantile convulsions. Other traditional uses include the treatment of diabetes, yellow fever and epilepsy. The phytochemistry of Nauclea Latifolia allowed the identification of alkaloids, mostly naucleamides as the main constituents.

Preferably, the roots are collected from the plant, dried and ground into powder. The process of the present invention is thus preferably carried out on a powder of roots with barks of Nauclea Latifolia. This step of grinding allows a better extraction since the specific surface area is higher.

Preferably, the process of extraction is carried out on root barks of Nauclea Latifolia.

### Step a)

In the present application, the term “appropriate solvent” is understood to mean a solvent appropriate for the extraction of the desired compound, (±)-cis-2-dimethylaminomethyl-1-(3-methoxyphenyl)cyclohexanol, that is to say capable of dissolving the compound to be extracted.

Preferred such solvents may be chosen among the alcoholic solvents and other solvents used for the extraction of organic molecules in the pharmaceutical industry. More particularly, preferred solvents are chosen among:
alcohols, preferably comprising from 1 to 6 carbon atoms, and more preferably from 1 to 4 carbon atoms, such as methanol, ethanol, n-propanol, isopropanol and n-butanol;
cetones, preferably comprising from 3 to 8 carbon atoms, and more preferably from 3 to 6 carbon atoms, such as acetone, methylethylcetone and butanone;
ethers, such as diethylether;
aromatic solvents, such as toluene and xylenes;
chlorinated solvents, such as chloroform, dichloromethane, trichloroethane, dichloroethene and trichloroethene; and
alkyl acetates, wherein the alkyl group preferably comprises from 1 to 6 carbon atoms, and more preferably from 1 to 4 carbon atoms, such as ethyl acetate, propyl acetate, isopropyl acetate, butyl acetate and isobutyl acetate.

According to a particular embodiment, the solvent is chosen in the group consisting of methanol, ethanol, n-propanol, isopropanol, chloroform, dichloromethane and ethyl acetate.

Preferably, the solvent is ethanol.

Typically, the step a) is carried out in a Soxhlet extractor.

The step a) is carried out for a sufficient time to extract essentially all the tramadol from the powder of roots of Nauclea Latifolia. Usually, the reflux is done for several hours, for example 24 hours.

After stopping the reflux, the solvent is evaporated to obtain a crude residue containing tramadol. The evaporation of the solvent may be done by distillation under reduced pressure.

At the end of step a), the crude residue obtained from the extraction contains tramadol along with other compounds extracted. Therefore, a step b) of purification is carried out to eliminate as much as possible the secondary compounds and to obtain tramadol with relatively high purity.

### Step b)

The purification of the crude residue may be done by well-known techniques of purification.

According to a particular embodiment, the step b) of purification comprises the following steps:
b1) treatment of the crude residue with an acid solution so as to transform cis-2-dimethylaminomethyl-1-(3-methoxyphenyl)cyclohexanol into an acid salt thereof,
b2) addition of an appropriate non water-miscible organic solvent S₁ to the acid solution thus obtained, leading to an acid aqueous phase A₁ containing said acid salt of cis-2-dimethylaminomethyl-1-(3-methoxyphenyl)cyclohexanol and an organic phase O₁,
b3) separation of said organic phase O₁ from said aqueous phase A₁,
b4) neutralization of said acid phase A₁ with an appropriate basic salt, leading to the neutral form of cis-2-dimethylaminomethyl-1-(3-methoxyphenyl)cyclohexanol,
b5) addition of an appropriate non water-miscible organic solvent S₂ so as to form an organic phase O₂ containing said neutral form of cis-2-dimethylaminomethyl-1-(3-methoxyphenyl)-cyclohexanol and an aqueous phase A₂, and
b6) separation of said organic phase O₂ from said aqueous phase A₂.
   Step b1)

Because of the presence of a nitrogen atom, cis-2-dimethylaminomethyl-1-(3-methoxyphenyl)cyclohexanol has basic properties.

The use of an acid solution transforms tramadol into an acid salt of tramadol and allows separating tramadol from other compounds present in the crude residue which do not have any basic property. Indeed, in the next step, an appropriate organic solvent is added and tramadol in the form of an acid salt will stay in the acid aqueous phase while the other compounds having no basic property will stay in the organic phase, leading to a first purification of the crude residue.

Typically, the step b1) is done by mixing the crude residue with a chlorhydric acid solution or a sulfuric acid solution.

According to a particular embodiment, the chlorhydric acid solution or the sulfuric acid solution comprises from 1 wt % to 20 wt % of chlorhydric acid or of sulfuric acid, respectively.

For example, the acid solution is an aqueous solution of HCl (5% in water).

The mixture of the crude residue and the acid solution is typically stirred at room temperature for several minutes, for example for 20 minutes.

Step b2)

As mentioned above, this step consists in adding an appropriate non water-miscible organic solvent S₁ in order to separate tramadol from the compounds having no basic property present in the crude residue.

When the organic solvent S₁ is added to the acid solution containing the crude residue, two phases are formed, an acid aqueous one, referred as A₁, and an organic one, referred as O₁. A₁ contains the formed acid salt of tramadol and O₁ contains the compounds having no basic property.

Typically, the organic solvent S₁ is a non water-miscible solvent chosen in the group consisting of dichloromethane, chloroform and ethyl acetate.

For example, the organic solvent S₁ is dichloromethane.

Step b3)

At the end of the step b2), the two phases A₁ and O₁ are separated and further steps of purification are carried out on the phase A₁ which contains tramadol.

Step b4)

Once the phase A₁ is collected, it is neutralized with a solution of an appropriate basic salt to return to neutral conditions, leading to the neutral form of cis-2-dimethylaminomethyl-1-(3-methoxyphenyl)cyclohexanol.

More precisely, the pH is increased in this step to a value approximately in the range from 6 to 7 so as to obtain the neutral form of tramadol which is soluble in organic solvents.

The basic salt may be chosen in the group consisting of sodium bicarbonate, sodium carbonate, potassium bicarbonate, potassium carbonate, sodium hydroxide and potassium hydroxide.

For example, the basic salt is sodium bicarbonate, used in the form of a saturated aqueous solution.

Step b5)

After returning to neutral conditions, an appropriate non water-miscible organic solvent S₂ is added, leading to an organic phase O₂ containing the neutral form of cis-2-dimethylaminomethyl-1-(3-methoxyphenyl)cyclohexanol obtained after step b4) and an aqueous phase A₂.

When the organic solvent S₂ is added to the neutralized aqueous phase solution containing tramadol, again two phases are formed, an aqueous one, referred as A₂, and an organic one, referred as O₂, which contains the neutral form of tramadol.

Typically, the organic solvent S₂ is a non water-miscible solvent chosen in the group consisting of dichloromethane, chloroform and ethyl acetate.

For example, the organic solvent S₂ is dichloromethane.

Usually, the mixture of the neutralized aqueous phase and the organic solvent S₂ is stirred at room temperature for a few minutes, for example 5 minutes.

Step b6)

The aqueous and organic phases are separated.

At the end of the step b6), tramadol is obtained in a relatively pure form in the organic phase O₂. In order to increase the purity as much as possible, several steps of washing and drying may be carried out.

According to a particular embodiment, the step b6) is followed by a step b7) of water washing and drying to eliminate traces of acid and basic impurities, leading to a purified residue containing (±)-cis-2-dimethylaminomethyl-1-(3-methoxyphenyl)-cyclohexanol.

Typically, the organic phase O₂ is washed with water, then dried over a drying agent, such as magnesium sulfate, and evaporated under reduced pressure.

At the end of the step b7), the purity of tramadol in the obtained residue is ranging from 80% to 90%, corresponding to a very satisfying purity.

According to another particular embodiment, the step b7) is followed by a step b8) of washing with diethyl ether and drying to eliminate traces of impurities, leading to a residue containing (±)-cis-2-dimethylaminomethyl-1-(3-methoxyphenyl)-cyclohexanol with increased purity.

Indeed, the inventors found that the use of diethyl ether allowed eliminating the small amounts of impurities left in the residue and thus increasing further the final purity.

Typically, the purified residue obtained after the step b7) is washed several times with diethyl ether, then diethyl ether is removed, for example by filtration, and the remaining residue is dried under vacuum for several hours, for example 24 hours.

After this step, tramadol is obtained as a thick yellow liquid.

Typically, the final purity of tramadol is ranging from 95% to 98%, which is a very good purity compared to prior art.

The yield of the process according to the invention may be upwards of 1%, notably ranging from 1% to 10%, and more particularly from 1% to 2% by weight of the total weight of roots of Nauclea Latifolia.

Consequently, the process of the present invention allows obtaining tramadol with high purity in a simple and cost-effective way.

The present invention also relates to an extract of Nauclea Latifolia obtainable by the process as defined above.

It also concerns an extract of Nauclea Latifolia obtainable by the process as defined above, comprising traces of alkaloids.

Indeed, several alkaloids are present in the roots of Nauclea Latifolia. In addition to 2-(dimethylaminomethyl)-1-(3-methoxyphenyl)cyclohexanol, the following alkaloids were also identified by LC/MS in the plant extract: nauclechine ([M+H]⁺=306.16), nauclefine ([M+H]⁺=288.11), vinconsamide ([M+H]⁺=499.21), and naucleamide E ([M+H]⁺=337.16) (FIG. 19). The presence of these alkaloids demonstrates that the plant extracts belong to Nauclea Latifolia.

The present invention also concerns a pharmaceutical composition comprising such an extract.

The present invention also relates to an extract of Nauclea Latifolia obtainable by the process as defined above, notably comprising traces of alkaloids, for use as a medicament.

It also concerns such an extract for use in the treatment of pain.

The anti-pain activity of the compound extracted by the process of the invention was assessed by several tests, namely the acetic acid induced abdominal constriction test, the formaline-induced nociception test, the hotplate test, the tail-flick test and the glutamate-induced nociception test, as described in Example 2 below.

These tests showed that the compound extracted by the process of the invention has an anti-nociceptive effect which is dose-dependent.

Besides, as described in Example 3 further, an interesting aspect is that 2-(dimethylaminomethyl)-1-(3-methoxyphenyl)cyclohexanol can be extracted from plants collected at different seasons, indicating the absence of marked seasonal disparity. Tramadol may thus be obtained year-round from Nauclea Latifolia.

### DESCRIPTION OF THE FIGURES

FIG. 1 is the experimental protocol scheme illustrating the extraction protocol used to identify the compound of interest, tramadol, in Nauclea Latifolia root barks.

FIG. 2 is the HPLC profile of the crude extract of Nauclea Latifolia root barks as a function of elution time in minutes. Major peaks are numbered and fractions (F25 to F29) investigated for the characterization of the anti-pain compound shown by the circle.

FIG. 3 represents the effects of fractions 25 to 29 of Nauclea Latifolia on the writhing induced by acetic acid. Results are expressed as mean±S.E.M (n=6). Statistical differences between control and treated groups were tested by two-way repeated measures analysis of variance (ANOVA), followed by Tukey's (HSD) multi-comparison A star (*) corresponds to P<0.05; three stars (***) to P<0.001 which corresponds to a significantly different result compared to the control groups.

FIG. 4 shows the influence of various concentrations of fraction 27 of Nauclea Latifolia on acetic acid-induced writhing, compared to aspirine and morphine, and the absence of counter effect of naloxone. Results are expressed as mean±S.E.M (n=6). Two stars (**) corresponds to P<0.01; three stars (***) to P<0.001 which corresponds to a significantly different result compared to the control groups. Abbreviations are ASA for aspirine and Morph for morphine.

FIG. 5 shows the influence of various concentrations of fraction 27 of Nauclea Latifolia on formalin-induced pain, compared to indomethacin and morphine. Results are expressed as mean±S.E.M. (n=6). A star (*) corresponds to P<0.05; two stars (**) to P<0.01; three stars (***) to P<0.001 which corresponds to a significantly different result compared to the control group. Abbreviations are Indom for indomethacin, Morph for morphine and Nalox for naloxone.

FIG. 6 shows the influence of various concentrations of fraction 27 of Nauclea Latifolia on hotplate-induced pain in mice, compared to aspirine and morphine. Results are expressed as mean±S.E.M. Abbreviations are ASA for aspirine, Morph for morphine and Nalox for naloxone.

FIG. 7 shows the influence of various concentrations of fraction 27 of Nauclea Latifolia on tail flick response in mice after immersion in 55° C. water bath, compared to aspirine and morphine. Results are expressed as mean±S.E.M. Abbreviations are ASA for aspirine, Morph for morphine and Nalox for naloxone.

FIG. 8 shows the influence of various concentrations of fraction 27 of Nauclea Latifolia on glutamate-induced pain, compared to dipyrone. Results are expressed as mean±S.E.M. (n=6). A star (*) corresponds to P<0.05; two stars (**) to P<0.01; ***, three stars (***) to P<0.001 which corresponds to a significantly different result compared to the control group.

FIG. 9 shows the chemical ionization mass spectrometry (CIMS) profile of the anti-pain compound from Nauclea Latifolia.

FIG. 10 shows the ¹H-NMR spectrum of the anti-pain compound from Nauclea Latifolia.

FIG. 11 shows the ¹³C-NMR spectrum of the anti-pain compound from Nauclea Latifolia.

FIG. 12 shows the COSY spectrum of the anti-pain compound from Nauclea Latifolia.

FIG. 13 shows the HMBC spectrum of the anti-pain compound from Nauclea Latifolia.

FIG. 14 shows the DEPT spectrum of the anti-pain compound from Nauclea Latifolia.

FIG. 15 is the chemical structure of the anti-pain compound showing the same structure as tramadol.

FIG. 16 shows the chiral HPLC profile of the extracted compound from Nauclea Latifolia.

FIG. 17 shows the ORTEP drawing of the two isomers.

FIG. 18 shows the chiral HPLC profiles of each purified tramadol enantiomer.

FIG. 19 shows the LC/MS profile of a crude extract from Nauclea Latifoli.

### EXAMPLES

### Example 1

### Process of extraction of tramadol from Nauclea Latifolia

Root bark of Nauclea Latifolia was collected from the National Park of Benoué (north Cameroon) in the dry season (April 2009). The plant was identified at the national herbarium (Yaoundé, Cameroon) where a voucher specimen (No. 20144/SRF/Cam) has been deposited. The fresh root bark collected was dried in an incubator at 65° C. and ground into powder.

20 g of powder of root bark were mixed with 300 ml of absolute ethanol and reflux was maintained for 24 hours in a Soxhlet system. Ethanol was distilled off under reduced pressure to yield a crude extract. The crude extract was mixed with an aqueous solution of HCl (5% in water) and stirred at room temperature for 20 minutes. The acidic solution was mixed with dichloromethane. The two phases (the aqueous and the organic phase) were separated. The aqueous solution was neutralized by addition of a saturated aqueous solution of sodium bicarbonate (NaHCO₃) until reaching a pH from 6 to 7. The obtained aqueous solution was mixed with dichloromethane. The obtained solution was stirred at room temperature for 5 minutes and the two phases (aqueous and organic) were separated. The organic solution was washed with water, dried over magnesium sulfate (MgSO₄) and evaporated under reduced pressure. The obtained residue was washed twice with diethyl ether (10 ml each time). The diethyl ether was removed by filtration and the remaining residue was dried under vacuum for 24 h to yield 200 mg of tramadol as a thick yellow liquid.

This corresponds to yield of 1% by weight compared to the weight of plant material. Purity of the product according to this protocol is >95% as evidenced by 1H NMR analysis.

### Example 2

### Bio-Guided Activity Screening on Extracts of Nauclea Latifolia Root Barks

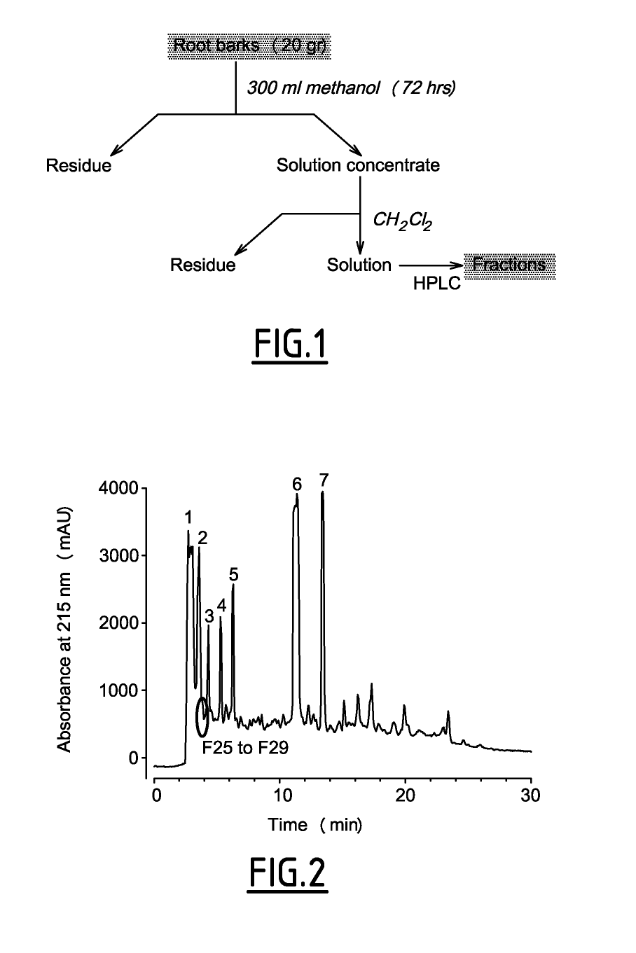

To isolate a compound active against pain, extracts of Nauclea Latifolia root barks were investigated through a bio-guided procedure (FIGS. 1 and 2).

A. Extraction of Nauclea Latifolia Root Barks

20 g of powdered plant material were mixed with 300 ml of methanol for 72 hours. The extract was filtered and the solvent was evaporated in a rotary evaporator under reduced pressure at 40° C. The residue was dissolved in 100 ml of dichloromethane and filtered. The solution was evaporated to dryness and solubilized in a 5% acetonitrile/95% water solution. Compounds were then separated by HPLC (column: C18, 250×10 mm, 10 μm, Vydac 218TP1010). The elution was performed using a 10-60% acetonitrile gradient for 40 min and yielded several fractions, notably fractions numbered 25 to 29 on FIG. 2, that were tested for anti-nociceptive activity. Elution speed was 10 ml/min, fraction size 500 μl, and fraction sampling was 20 fractions/min.

B. Animal Experimentation

Experiments in mice were performed on C57BL/6 male mice (Janvier, Le-Genest-St-Isle, France) weighting 26-35 g, housed in individual cages with food and water ad libitum and kept in a 12 hours/12 hours light-dark cycle. All animal experimentations were carried out in accordance with the rules of the European Committee Council Directive of Nov. 24, 1986 (86/609/EEC) and all procedures were approved by the local department of the veterinarian services for the use and care of animals (agreement #380612). All efforts were made to minimize animal suffering and reduce the number of animals used in each series of experiments.

C. Bio-Guided Activity Screening

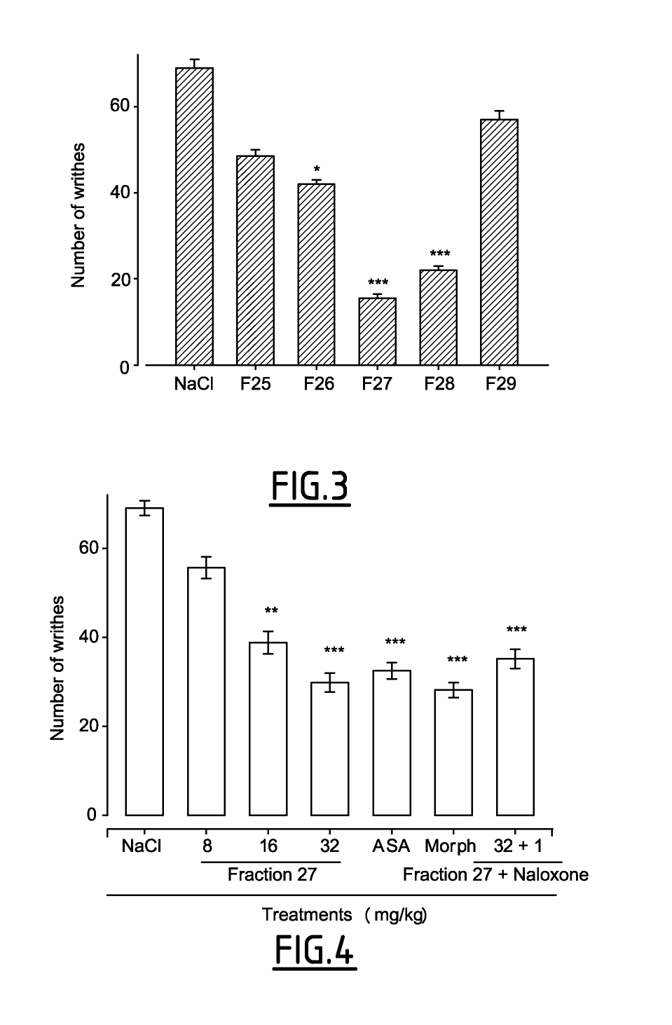

Using the writhing test in rats, it was showed that active anti-pain compounds were present in the final extract of the root barks. Further HPLC fractionation of this extract indicates that the bioactivity resides within fractions N °25 to 29, with a peak of activity in fraction 27 (FIGS. 2 and 3). All other fractions were inactive with regard to the writhing test in rats. The analgesic activity of fraction N °27 was evaluated using several nociceptive tests. Thin layer chromatography (TLC) analysis and ¹H NMR showed that fraction 27 contains a single compound with purity over 95%.

D. Acetic Acid Induced Abdominal Constriction Test

The methanolic fraction of Nauclea Latifolia (16, 40 or 80 mg/kg, per oral (p.o.)) or HPLC fractions (8, 16 or 32 mg/kg, p.o.), purified tramadol isolated from Nauclea Latifolia (8, 16 or 32 mg/kg, p.o.), aspirin (150 mg/kg, p.o.), morphine (5 mg/kg, s.c.), naloxone+methanolic fraction (respectively 1 mg/kg, intraperitoneally (i.p.)+80 mg/kg, p.o.), naloxone+HPLC fraction (respectively 1 mg/kg, i.p.+32 mg/kg, p.o.) or 0.9% NaCl (p.o.) were administered one hour prior to treatment with acetic acid.

One hour after oral administration of these substances, each animal was injected i.p. with 0.6% acetic acid in a volume of 10 ml/kg body weight. After acetic acid injection, the number of stretching or writhing responses per animal was recorded during 30 min after a latency period of 5 min. Inhibition was expressed in percentage.

The fractions began manifesting their assuaging effects on the writhing reflex 45 min following administration. Statistical analyses were thus performed on data obtained 45 min following administration of fraction 27. Data were analyzed by two-way Anova followed by Tukey's (HSD) multi-comparison test.

The results of the acetic acid-induced abdominal constriction test are shown in Table 1.

TABLE 1
Acid-induced abdominal construction test

| | | Acetic acid- |
|---|---|---|
| | Doses | induced writhing |
| Treatments | (mg/kg) | inhibition (%) |
| NaCl | — | — |
| F27 of N. latifolia | 8 | 19.3 |
| F27 of N. latifolia | 16 | 43.7 |
| F27 of N. latifolia | 32 | 56.8 |
| Aspirin | 150 | 52.9 |
| Morphine | 5 | 59.2 |
| F27 of N. latifolia + Naloxone | 32 + 1 | 49.0 |

As shown on FIG. 4, fraction 27 produced a dose-dependent (8, 16 or 32 mg/kg, p.o.) inhibition of the acetic acid-induced abdominal constrictions in mice.

The mean ID₅₀ value for oral administration of fraction 27 (and its respective 95% confidence limits) and the maximal inhibition were 14.13 (5.53-41.88) mg/kg and 56.8% [F(6, 78)=101.42; p<0.001].

E. Formaline-Induced Nociception

The formalin test was carried out as described by Hunskaar and Hole (1987) with slight modifications. The negative control was treated with 0.9% NaCl. The positive control received indomethacin (10 mg/kg, p.o.) or morphine (5 mg/kg, subcutaneously (s.c.)), two reference analgesic compounds. Other groups of mice were treated with methanolic fractions of Nauclea Latifolia (16, 40 or 80 mg/kg, p.o.), or purified tramadol isolated from Nauclea Latifolia (8, 16 and 32 mg/kg, p.o.). Pain was induced by injecting 50 μl of 2.5% formalin (40% formaldehyde in distilled water) in the right hind paw pad. Mice were given different substances 1 hour prior formalin injection. Animals were individually placed in a transparent Plexiglas cage (27×20×18 cm) observation chamber. The amount of time spent licking and biting the injected paw was indicative of pain and was recorded during the first 0-5 min (first phase), followed by the 15-30 min period (second phase). Data were analyzed by two-way Anova followed by Tukey's (HSD) multi-comparison test.

Purified fraction 27 isolated from Nauclea Latifolia had analgesic effects on both first (0-5 min) and second phases (15-30 min) of formalin test as shown in FIG. 5. These phases corresponded to neurogenic and inflammatory pains, respectively. Its neurogenic-induced pain blockade occurred at 32 mg/kg (64.07%, [F(6, 27)=95.17; p<0.001] whereas beginning from 8 mg/kg. Similarly, the extract at the dose of 32 mg/kg significantly blocked pain emanating from inflammation (31.74%) [F(6, 27)=95.17; P<0.05].

F. Hotplate Test

The apparatus consisted of a water bath in which was placed a metallic cylinder (14 cm diameter×10 cm height). The hot plate was maintained at 55±0.5° C. Each mouse (six per group) acted as its own control. One hour before treatment, the reaction time of each mouse (liking of the forepaws or jumping response) was measured twice with a 10 min interval. The average of the two readings was obtained as the initial reaction time. The reaction time following the administration of purified tramadol isolated from Nauclea Latifolia (8, 16 or 32 mg/kg, p.o.), aspirine (100 mg/kg, p.o.), morphine (5 mg/kg, s.c.), naloxone+HPLC fraction (1 mg/kg, i.p.+32 mg/kg, p.o.) and 0,9% NaCl (p.o.) was measured at 0.5, 1, 5 and 6 hours after a latency period of 30 min.

The protection percentage was calculated as the ratio (reaction time following tramadol administration−initial reaction time): initial reaction time.

The results of the hotplate test are shown in Table 2.

TABLE 2
Hotplate test

| | Doses | Protection against thermal nociception (%) | | | | | | | |
|---|---|---|---|---|---|---|---|---|---|
| Treatments | (mg/kg) | 0 hr | 0.5 hr | 1 hr | 2 hrs | 3 hrs | 4 hrs | 5 hrs | 6 hrs |
| NaCl | — | — | −6.6 | −2.2 | 3.9 | −0.6 | 10.0 | 3.3 | 9.6 |
| F27 of N. latifolia | 8 | — | −2.2 | 22.9 | 20.2* | 50.6** | 4.6 | −0.2 | −0.5 |
| F27 of N. latifolia | 16 | — | 1.9 | 27.0* | 21.3* | 52.4** | 6.5 | −2.0 | 4.6 |
| F27 of N. latifolia | 32 | — | 1.9 | 45.2** | 50.9** | 71.9*** | 25.2* | 21.3* | 27.8* |
| Aspirin | 150 | — | 1.7 | 35.5* | 2.2* | 39.5* | 8.5 | 10.6 | 3.5 |
| Morphine | 5 | — | 7.4 | 44.4** | 46.8** | 40.4** | 43.6** | 24.6* | 17.1 |
| F27 of N. latifolia + | 32 + 1 | — | −2.1 | 33.8* | 30.6* | 58.9** | 42.4** | 23.7* | 27.7** |
| Naloxone | | | | | | | | | |

The extract, at all doses used, began manifesting its assuaging effect on the writhing reflex 1 hr following administration.
*P < 0.05;
**P < 0.01;
***P < 0.001, significantly different compared to the control group.
Data were analysed by two-way Anova followed by the Tukey's (HSD) multi-comparison test.

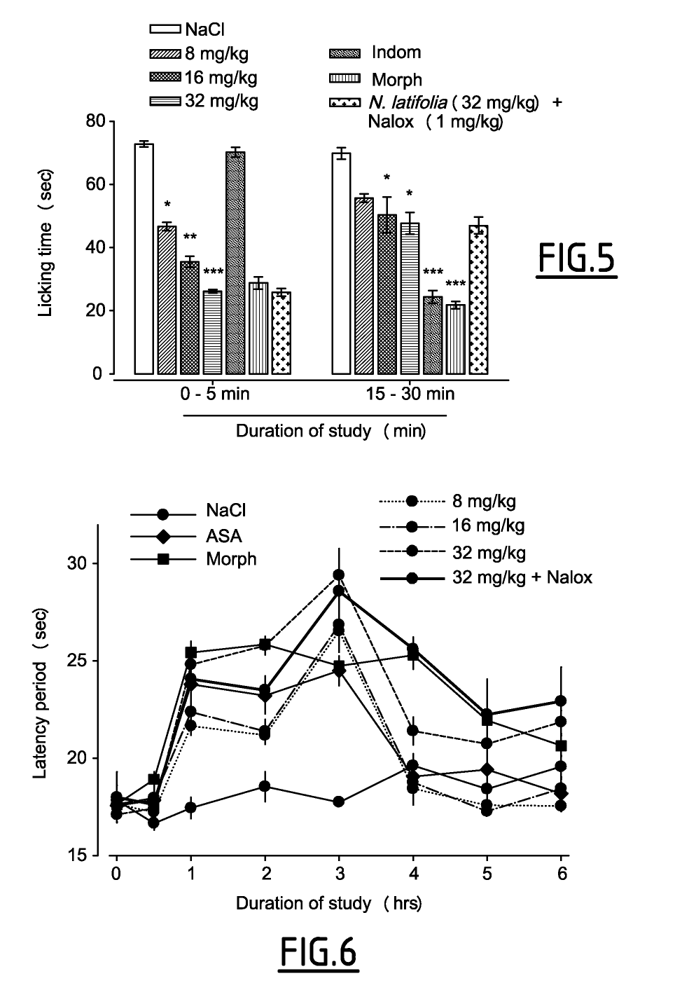

FIG. 6 shows that the purified fraction isolated from Nauclea Latifolia tested marked increase in the latency response in the hot plate algesiometer model of nociception, with the higher dose administered (32 mg/kg) and the maximal effect was observed in later times after oral administration (1-3 hours). In this regard, since 60 min after its oral administration it could be observed a significant increase [F(6, 43)=127.05; p<0.001] in baseline that reached maximal level at 3 hours. Naloxone antagonized antinociceptive effect of the purified tramadol isolated from Nauclea Latifolia in hot plate assay procedures.

G. Tail-Flick Test

The tail-flick test was carried out according to the method described by D'Amour and Smith. This involved immersing extreme 3 cm of the mice's tail in water bath containing water at a temperature of 55±0.5° C. Within a few seconds, the mice reacted by withdrawing the tail. The reaction time was recorded with a stopwatch. The mice were treated with purified tramadol isolated from Nauclea Latifolia (8, 16 or 32 mg/kg, p.o.), aspirine (100 mg/kg, p.o.), morphine (5 mg/kg, s.c.), naloxone+HPLC fraction (1 mg/kg, i.p.+32 mg/kg, p.o.) and 0.9% NaCl (p.o.). The reaction time of mice was taken at intervals of 15, 30 and 60 min after a latency period of 1 hour following the administration of the decoction and drugs.

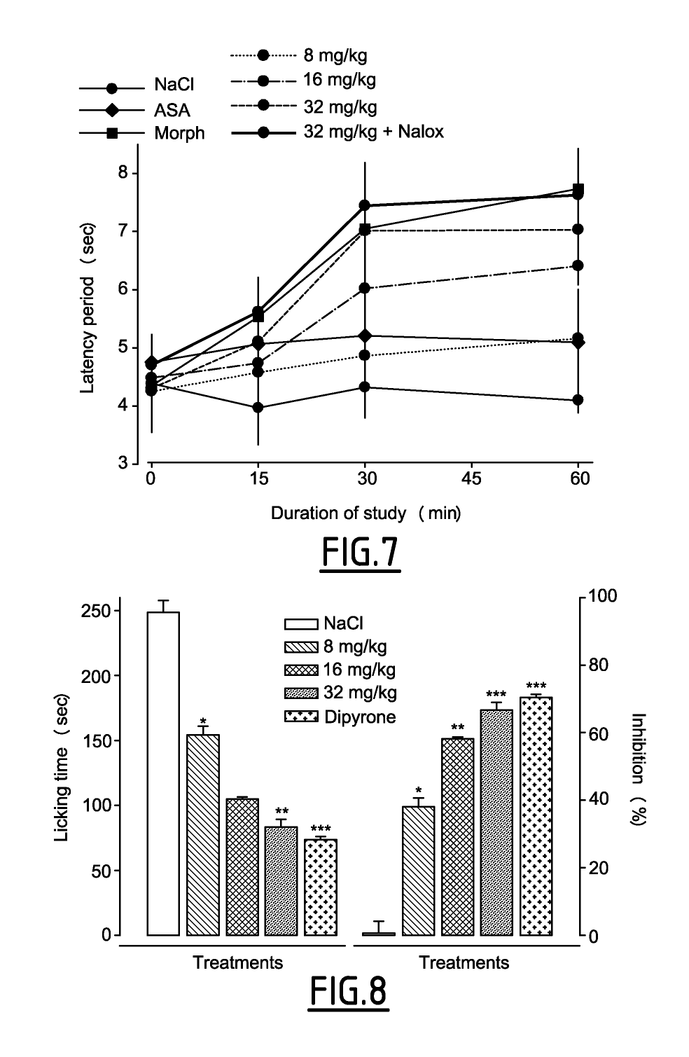

After a latency period of 30 min following oral administration of fraction 27 isolated from Nauclea Latifolia at a dose of 32 mg/kg (63.05%), [F(6, 57)=97.24; p<0.001], there was a significant reduction of painful sensation due to tail immersion in warm water and it was dose-dependent (see FIG. 7). The inhibitory effects of fraction 27 became pronounced between 30 and 60 post-dosing and reached a maximum of 63.5% [F(6, 57)=97.24; p<0.001] with the dose of 800 mg/kg. The analgesic activity of the extract was blocked by naloxone.

The protection percentage was calculated as the ratio (reaction time following tramadol administration−initial reaction time): initial reaction time.

The results of the tail-flick test are shown in Table 3.

TABLE 3
Tailflick test

| | | Protection agains thermal | | | |
|---|---|---|---|---|---|
| | Doses | nociception (%) | | | |
| Treatments | (mg/kg) | 0 min | 15 min | 30 min | 60 min |
| NaCl | — | — | −18.8 | −11.6 | −16.1 |
| F27 of N. latifolia | 8 | — | 7.7 | 14.4* | 21.4* |
| F27 of N. latifolia | 16 | — | 5.6 | 34.3** | 42.9*** |
| F27 of N. latifolia | 32 | — | 18.7 | 63.0*** | 63.5*** |
| Aspirin | 150 | — | 6.4 | 9.5 | 7.1 |
| Morphine | 5 | — | 27.3 | 62.1*** | 77.8*** |
| F27 of N. latifolia + | 32 + 1 | — | 19.5 | 58.3*** | 62.2*** |
| Naloxone | | | | | |

Results are expressed as percentage of protection against thermal nociception.
Statistical analyses were performed on absolute data, n = 6.
*, P < 0.05;
**, P < 0.01;
***, P < 0.001 significantly different compared to the control group.
Data were analyzed by two-way Anova followed by Tukey's (HSD) multi-comparison test.

H. Glutamate-Induced Nociception

Animals were treated with the purified tramadol isolated from Nauclea Latifolia (8, 16 or 32 mg/kg, p.o.), dipyrone (60 mg/kg, p.o.) or 0.9% NaCl (p.o.) 1 hour before test. A volume of 20 μl of glutamate (30 μmol/paw) was injected intraplantarly in the ventral surface of the right hind paw. Animals were observed individually for 15 min following glutamate injection. The amount of time they spent licking the injected paw was recorded with a chronometer and was considered as indicative of nociception. Data were analyzed by two-way Anova followed by Tukey's (HSD) multi-comparison test.

Interestingly in the glutamate induced nociception in mice, fraction 27 isolated from Nauclea Latifolia, caused marked and dose-related antinociception (FIG. 8). The calculated mean ID₅₀ values (and it's 95% confidence limits) and the maximal inhibition were 21.17 (6.49-47.23) and 66.55% [F(4, 62)=105.12; p<0.001] respectively. Given orally, dipyrone produced significant inhibition of 70.30% [F(4, 62)=105.12; p<0.001] of the glutamate-induced nociception in mice.

Consequently, the fraction 27 isolated from Nauclea Latifolia showed significant efficacy on the different nociceptive tests, indicating that it contains an active anti-pain compound.

### Example 3

### Chemical Characterization of the Anti-Pain Compound from Nauclea Latifolia

The chemical characterization of the tramadol was done by several techniques, namely high resolution chemical ionization mass spectrometry (HRCIMS), ¹H-NMR, ¹³C-NMR, two-dimensional NMR data ¹H-¹-H correlation spectroscopy (COSY), heteronuclear multiple bond connectivity (HMBC), distortion-less enhancement by polarization transfer (DEPT), LC/MS, chiral HPLC and X-ray diffraction, as described in Example 3 further.

These different techniques allowed determining the chemical structure and the stereochemistry of the compound extracted by the process of the invention, and thus confirming that it was actually tramadol.

A. Material and Methods

NMR spectra were recorded on a Bruker AC-400 instrument (400 MHz). Chemical shifts (δ) are reported in ppm relative to Me₄Si (internal standard). Electrospray ionization ESI mass spectra on an Esquire 300 Plus Bruker Daltonis instrument with a nanospray inlet. Combustion analyses were performed, and all tested compounds have a purity of at least 95%. Thin-layer chromatography (TLC) used Merck silica gel F-254 plates (thickness 0.25 mm). Flash chromatography used Merck silica gel 60, 200-400 mesh. Unless otherwise stated, reagents were obtained from commercial sources and were used without further purification. Commercial tramadol was purchased from Sigma.

B. Determination of the Chemical Structure

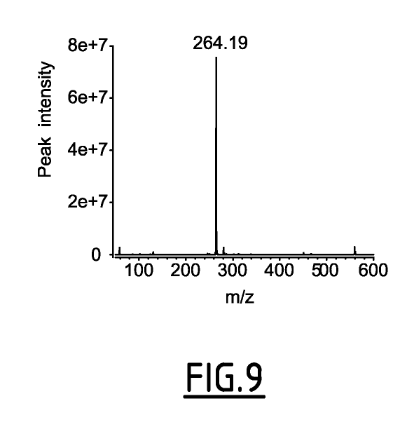

The purified product showed the appearance of a transparent and oily substance. The purified compound was optically inactive and characterized as a racemic mixture. High resolution chemical ionization mass spectrometry (HRCIMS) of the compound shows that it has an experimental m/z [M+H]⁺ of 264.19 and its molecular formula was deduced as C₁₆H₂₅NO₂ (FIG. 9).

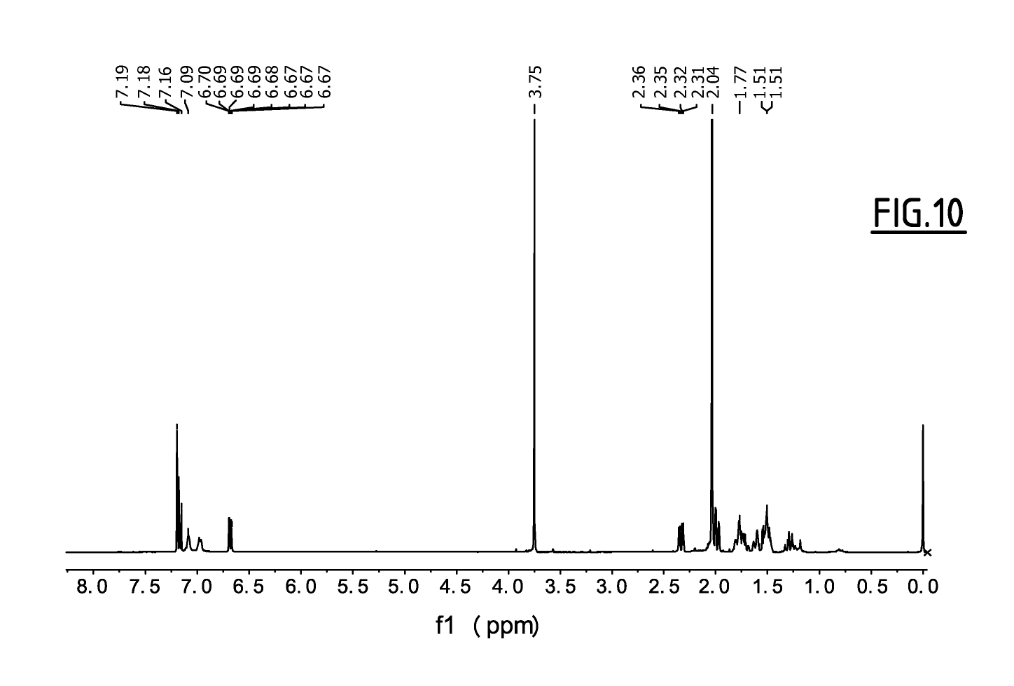

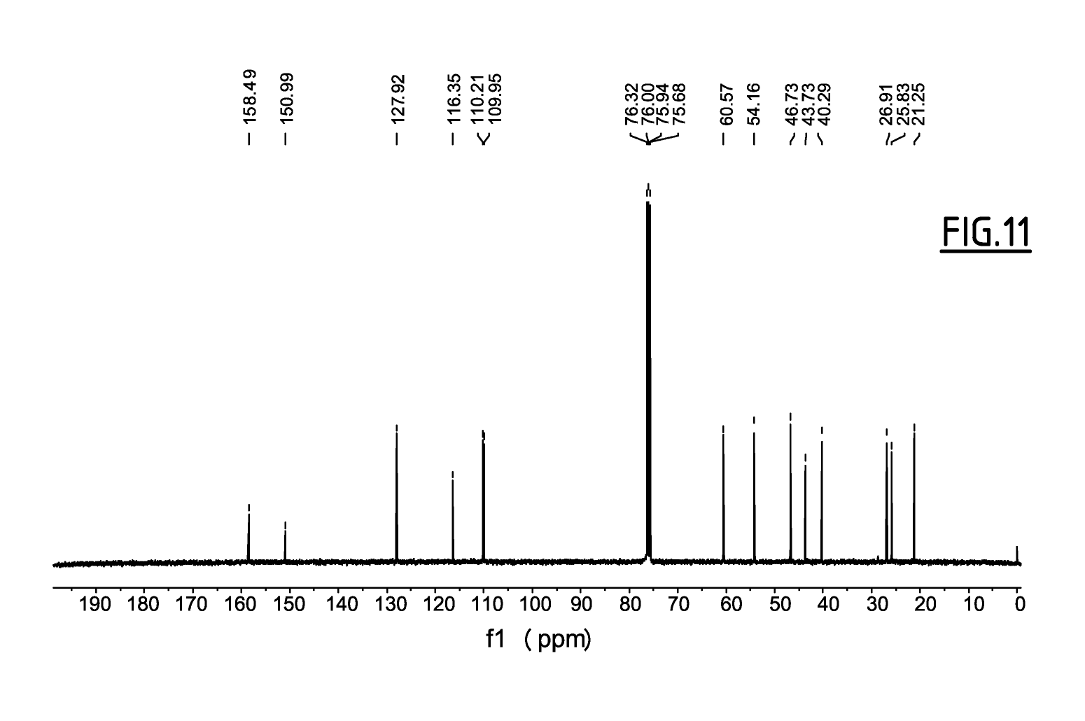

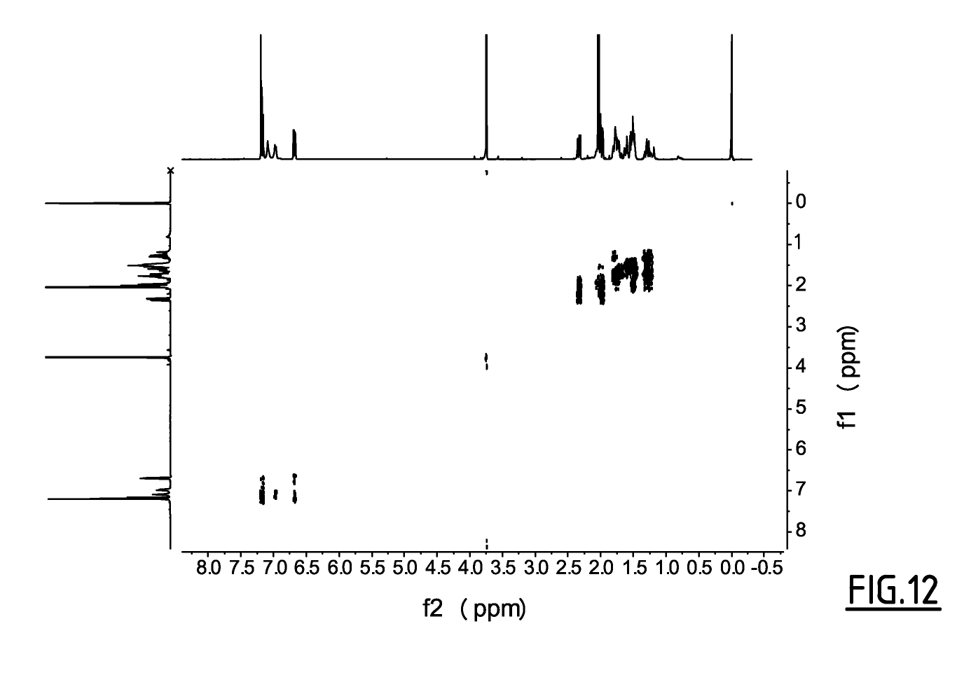

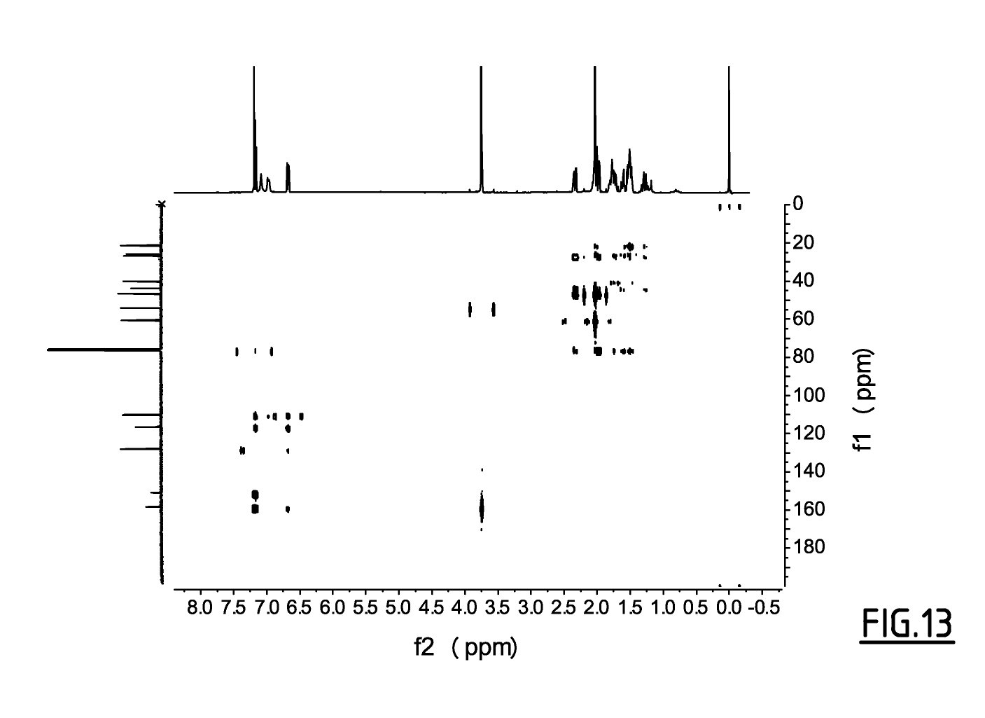

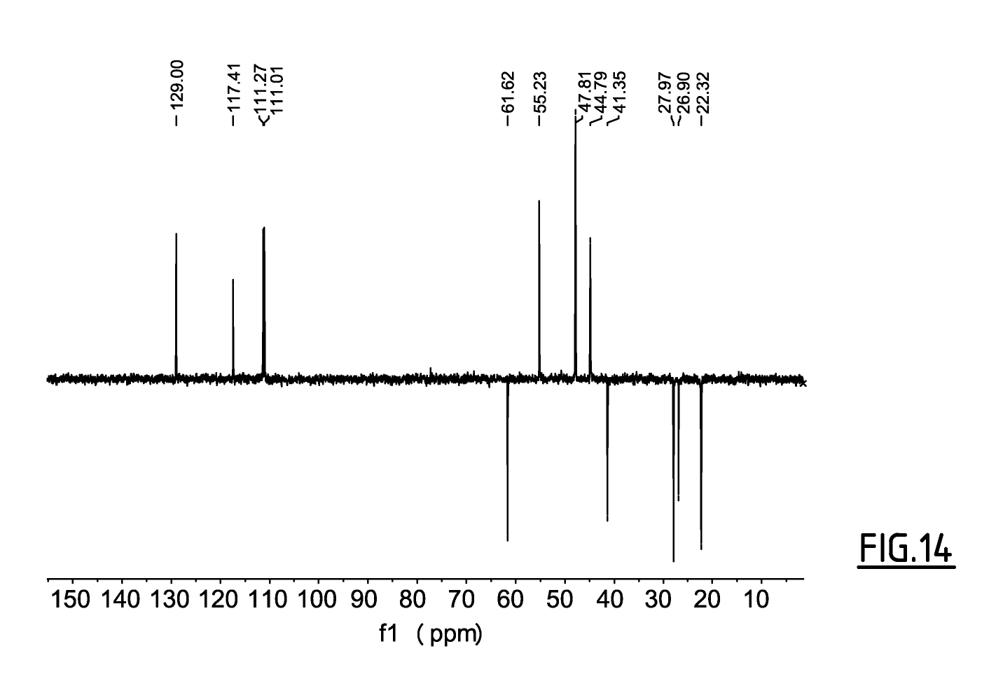

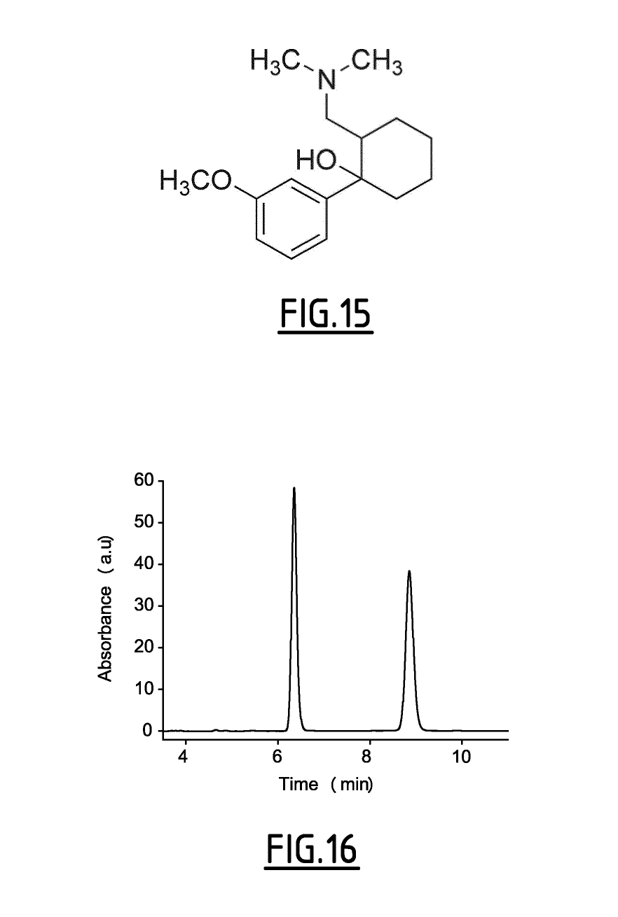

The full structure of the compound was deduced from detailed analyses of ¹H-NMR (FIG. 10) and ¹³C-NMR (FIG. 11) data together with two-dimensional NMR data ¹H-¹H correlation spectroscopy (COSY) (FIG. 12) and heteronuclear multiple bond connectivity (HMBC) (FIG. 13). The ¹³C-NMR indicates the presence of 15 signals corresponding to at least 15 carbon atoms. Spectroscopic data from distortion-less enhancement by polarization transfer (DEPT) indicates the presence of five CH₂, three CH₃ or aliphatic CH, and four aromatic CH carbons (FIG. 14). From the HMBC experiment, it was deduced that two out of three methyl groups are linked to the same atom (FIG. 13). The ¹H-NMR spectrum exhibited four aromatic proton resonances δH 6.76 (1H, dd, J₁=2.24 Hz, J₂=8.08 Hz), 7.03 (1H, bd, J=7.16 Hz, H5), 7.13 (1H, bs), and 7.24 (1H, t, J=8.08 Hz) indicating a 1,3-disubstituted aromatic ring. The presence of a methoxy group was evidenced by the presence of a singlet at 3.77 ppm and a signal at 56.2 ppm in ¹H-NMR and ¹³C-NMR, respectively. The complex spin-system at high field δH 1.35-2.65 suggested the presence of cyclic alkyl chain in the molecule. The quaternary carbon at 76.54 ppm was assigned to an oxygenated carbon. The linkage of the latter to the aromatic ring was evidenced by its correlation to the C-2′ carbon. The structure of the molecule as determined by these spectroscopic and spectrometric data, shown in FIG. 15, is 2-(dimethylaminomethyl)-1-(3-methoxyphenyl)cyclohexanol. The structure was confirmed by the crystal structure X-ray diffraction analysis. Interestingly, the chemical structure of this molecule matches that of tramadol.

Compound 2-(dimethylaminomethyl)-1-(3-methoxyphenyl)cyclohexanol was reliably detected and purified from three different plant samples collected in the National Park of Benoué (North Cameroon) at different seasons, indicating the absence of marked seasonal disparity.

C. Determination of the Isotopic Content of 2-(Dimethylaminomethyl)-1-(3-Methoxyphenyl) Cyclohexanol from Nauclea Latifolia

Finally, a complete comparison of the isotopic content of natural 2-(dimethylaminomethyl)-1-(3-methoxyphenyl)cyclohexanol and two different commercial sources of tramadol (pure from Sigma or extracted from commercialized drug from Sanofi-Aventis) were performed.

The samples were dissolved in MeOH and an aliquot containing approximately 6 mg of compound was pipetted into each of two tin capsules (solids “light” 5×9 mm, Thermo Fisher Scientific). After evaporation of solvent with N₂ gas, the precise mass was measured.

The ¹³C/¹²C and ¹⁵N/¹⁴N ratios were obtained by isotope ratio measurement by mass spectrometry (irm-MS) using a Sigma2 spectrometer (Sercon Instruments, Crewe, UK) linked to a Sercon elemental analyser. Isotope ratios (δ¹³C and δ¹⁵N (%)) were expressed relative to the international references using the following equation:

$$δ ( \% o ) = ( \frac{R}{{R}_{std}} - 1 ) \times 1000$$

where for δ¹³C the reference (R_{std}) is Vienna-Pee Dee Belemnite (V-PDB) and for δ¹⁵N it is atmospheric N₂. The calibrated international reference materials NBS-22, SUCROSE-C6, and PEF-1 (IAEA, Vienna, Austria) were used for δ¹³C calibrations, via a laboratory standard of glutamic acid. The calibrated international reference materials IAEA-N1 or IAEA-N2 (IAEA, Vienna, Austria) were used for δ¹⁵N calibrations, via a laboratory standard of glutamic acid.

The ¹⁵N/¹⁴N and ¹³C/¹²C isotope ratios in two extracts of natural tramadol and in four samples of tramadol obtained from two different commercial sources were compared. The results are presented in Table 4.

TABLE 4
Isotope ratios ¹⁵N/¹⁴N and ¹³C/¹²C

| Sample | δ¹⁵N (%°) | range | δ¹³C (%°) | range |
|---|---|---|---|---|
| Commercial 1-1 | −2.61 | 0.05 | −29.97 | 0.15 |
| Commercial 1-2 | −9.24 | 0.84 | −29.73 | 0.20 |
| Commercial 1-3 | 5.68 | 0.30 | −29.61 | 0.12 |
| Commercial 2-1 | −1.79 | 0.10 | −31.97 | 0.10 |
| Natural 1 | −3.22 | 0.45 | −32.68 | 0.10 |
| Natural 2 | −3.13 | 0.23 | −31.98 | 0.04 |

It was found that the natural compound differs from tramadol by isotopic content. Negligible differences can be seen between the natural and commercial samples on the basis of the δ¹³C % values. In contrast, a range of values are found for the δ¹⁵N values, depending on the source. The wide range of ¹⁵N/¹⁴N ratios in the commercial samples of tramadol probably reflects the use of different batches of methylamine for synthesis. While the natural samples do not fall in a distinctly different range of values, it is notable that they are both very similar in isotope ratio and significantly different from all the values obtained for the commercial samples.

D. Stereochemistry Elucidation of 2-(Dimethylaminomethyl)-1-(3-Methoxyphenyl) Cyclohexanol from Nauclea Latifolia

According to the chemical structure, there are four possible existing stereoisomers of 2-(dimethylaminomethyl)-1-(3-methoxyphenyl)cyclohexanol that may be present in bark roots of Nauclea Latifolia. The isolated compound has no optical activity as evidenced by optical rotation measurements ([a]_{D}=0). This indicates that the isolated compound is racemic. Therefore, the stereochemistry of the isolated compound was to be determined.

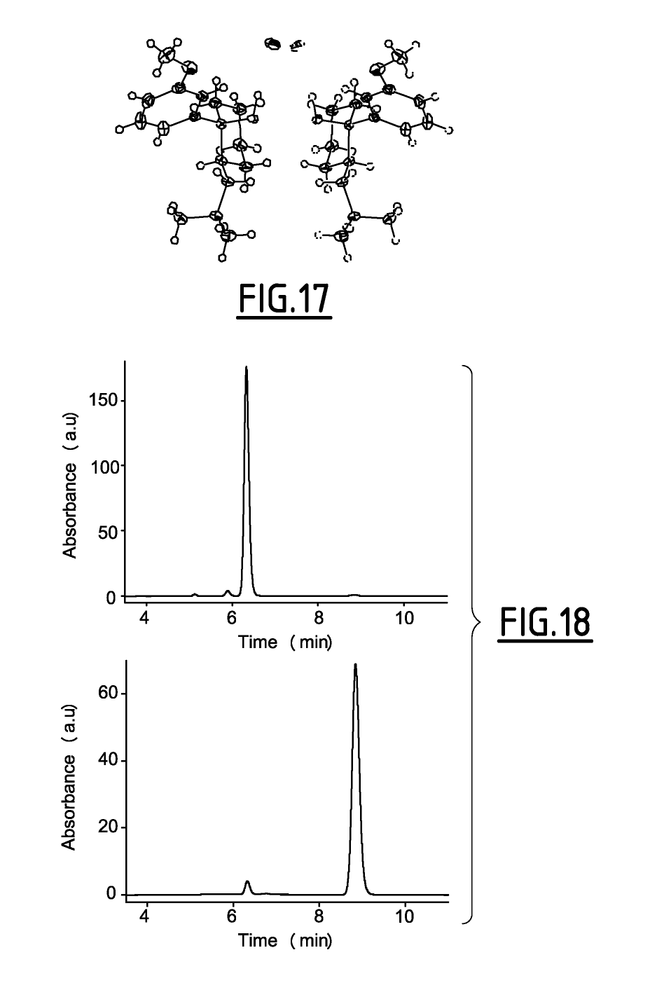

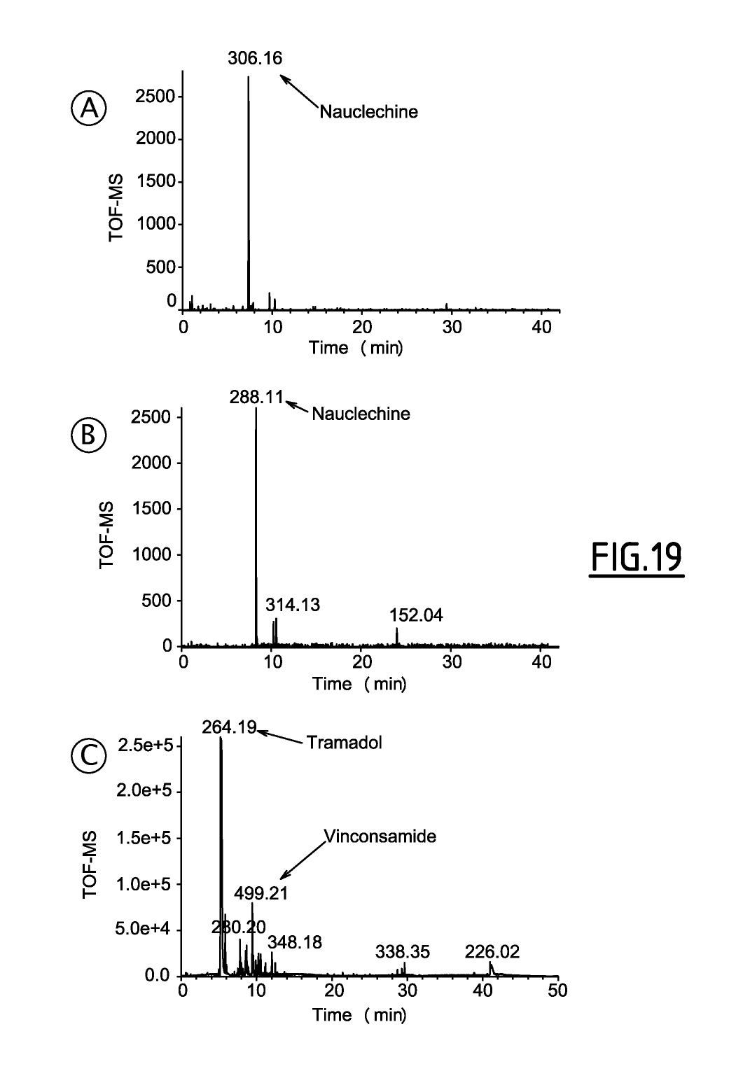

First, chiral HPLC was performed on the extracted material and showed the presence of two peaks indicating the presence of two isomers in equal amounts (FIG. 16). Second, the isolated compound was transformed to a hydrochloride salt and crystallized in acetonitrile. X-ray diffraction analyses confirmed the chemical structure and provided the stereochemistry of the natural compound (FIG. 17). In addition, these data illustrate the presence of two isomers of cis-2-(dimethylaminomethyl)-1-(3-methoxyphenyl)cyclohexanol: (R,R)-cis-2-(dimethylaminomethyl)-1-(3-methoxyphenyl) cyclohexanol and (S,S)-cis-2-(dimethylaminomethyl)-1-(3-methoxyphenyl)cyclohexanol (FIG. 17). Interestingly, these two isomers are the same isomers found in the composition of commercial tramadol. Each one of these enantiomers of the commercialized tramadol was chemically separated by racemic separation according to a well-established protocol (Evans, G. R., Henshilwood, J. A., O'Rourke, J. (2001) Tetrahedron: Assymetry 12, 1663-1670). These enantiomers were then run on chiral HPLC and their elution profiles found to be identical to those observed with the extracted compound (FIG. 18). The presence of these two isomers in Nauclea Latifolia extracts can be explained in two ways: i) the plant produces both isomers or ii) epimerization from one isomer towards the other one occurs as a consequence of the extraction protocol.

1. Process for obtaining (±)-cis-2-dimethylaminomethyl-1-(3-methoxyphenyl)-cyclohexanol comprising the following steps:
a) extracting from roots of Nauclea Latifolia with an appropriate solvent, leading to a crude residue, and
b) purifying said crude residue to obtain a purified residue containing (±)-cis-2-dimethylaminomethyl-1-(3-methoxyphenyl)cyclohexanol.

2. Process according to claim 1, wherein the solvent of step a) is selected from the group consisting of methanol, ethanol, n-propanol, isopropanol, chloroform, dichloromethane and ethyl acetate.

3. Process according to claim 1, wherein the solvent of step a) is ethanol.

4. Process according to claim 1, wherein the step b) comprises the following substeps:
b1) treating the crude residue with an acid solution so as to transform cis-2-dimethylaminomethyl-1-(3-methoxyphenyl)cyclohexanol into an acid salt thereof,
b2) adding of an appropriate non water-miscible organic solvent S₁ to the acid solution thus obtained, leading to an acid aqueous phase A₁ containing said acid salt of cis-2-dimethylaminomethyl-1-(3-methoxyphenyl)cyclohexanol and an organic phase O₁,
b3) separating said organic phase O₁ from said aqueous phase A₁,
b4) neutralizing said acid phase A₁ with an appropriate basic salt, leading to the neutral form of cis-2-dimethylaminomethyl-1-(3-methoxyphenyl)cyclohexanol,
b5) adding an appropriate non water-miscible organic solvent S₂ so as to form an organic phase O₂ containing said neutral form of cis-2-dimethylaminomethyl-1-(3-methoxyphenyl)cyclohexanol and an aqueous phase A₂, and
b6) separating said organic phase O₂ from said aqueous phase A₂.

5. Process according to claim 4, wherein in step b1), the acid solution is a chlorhydric acid solution or a sulfuric acid solution.

6. Process according to claim 4, wherein the organic solvents S₁ and S₂, identical or different, are selected from the group consisting of dichloromethane, chloroform and ethyl acetate.

7. Process according to claim 4, wherein the organic solvents S₁ and S₂ are dichloromethane.

8. Process according to claim 4, wherein in step b4), the basic salt is selected from the group consisting of sodium bicarbonate, sodium carbonate, potassium bicarbonate, potassium carbonate, sodium hydroxide and potassium hydroxide.

9. Process according to claim 1, wherein the roots of Nauclea Latifolia are in the form of a powder.

10. Process according to claim 1, wherein the roots of Nauclea Latifolia are root barks.

11. Extract of Nauclea Latifolia obtainable by the process according to claim 1.

12. Extract according to claim 11, comprising alkaloids.

13. Pharmaceutical composition comprising the extract according to claim 11.

14. A method of treating a subject comprising administering the extract according to claim 11 to said subject.

15. A method of treating pain in a subject in need thereof comprising administering to said subject the extract according to claim 11.
